(19) United States
(12) Patent Application Publication
(10) Pub. No.: US 2014/0287064 A1
(43) Pub. Date: Sep. 25, 2014

(54) Compositions for improving the appearance and/or treating fungal infections of nails, mucus membranes and the integument

(71) Applicant: Karen G. Swenholt, Annandale, VA (US)

(72) Inventor: Karen G. Swenholt, Annandale, VA (US)

(21) Appl. No.: 14/120,510

(22) Filed: May 28, 2014

Related U.S. Application Data
(63) Continuation-in-part of application No. 13/507,871, filed on Aug. 3, 2012.
Application No. 13/507,871 is a continuation-in-part of application No. 12/587,495, filed on Oct. 7, 2009.
Application No. 12/587,495 is a continuation-in-part of application No. 12/387,119, filed on Apr. 28, 2009, now abandoned.

(51) Int. Cl. A61K 33/20; A61K 36/87; A61K 45/06; A61K 31/05; A61K 36/534; A61K 36/63; A61K 36/53

(52) U.S. Cl. CPC A61K 33/20; A61K 36/63; A61K 36/87; A61K 36/53; A61K 31/05; A61K 36/534; A61K 45/06
USPC 424/665

(57) ABSTRACT

Fungal infections and other disease conditions of the integument can be effectively treated with compositions containing ozonizable oils, plant extract oils and antimicrobials, when used in conjunction with solutions containing sodium chlorite. The compositions are particularly useful for treatment of resistant fungal infections.

This application claims priority from Provisional Patent Application 61/573,008 filed Aug. 4, 2011 and from Provisional Patent Application 61/663,993 filed Feb. 22, 2012 This application claims priority from U.S. patent application Ser. No. 13/507,871, filed Aug. 3, 2012 and U.S. patent application Ser. No. 12/587,495 filed Oct. 7, 2009. The contents of each of these applications in incorporated in its entirety.

### BACKGROUND OF THE INVENTION

The present invention relates generally to methods and compositions for the treatment of dermatological conditions in humans. It is particularly efficacious for treating infection-induced conditions of the integument and is particularly well adapted for us in treating fungal infections of the finger and toenails.

Damage to the integument is from many sources. Many adverse conditions arise from exposure to infections of various types. About 7-10% of Americans suffer from onychomycosis, a difficult to treat a fungal infection of the nail, particularly the toenail. More than 90 percent of cases are caused by one of two pathogens: Trichophyton rubrum or Trichophyton mentagrophytes, which is also primarily responsible for tinea pedis. Factors that have an important effect on the development of onychomycosis include increasing age; genetic susceptibility; and the presence of certain disease states such as diabetes, acquired immunodeficiency syndrome, or peripheral arterial disease. All of the disease conditions of the integument are exacerbated when other disease conditions such as diabetes are present. For example, it is reported that up to one third of diabetics develop nail fungus. In the presence of any disease affecting circulation there is slowing of healing, particularly in the feet. The compositions of the invention were first developed to treat such infections, but are also useful for treating many other conditions of the integument such as acne and other skin blemishes that arise from pressure, stress or infection.

Hoofs of horses and claws of dogs and cats contain keratin and are hard like human nails. Animals, particularly mammals can benefit from these treatments against dermatological infections and infections of hoofs and claws.

Feet exposed to a warm, dark, moist environment can get infected. Some people may already be genetically predisposed to onychomycosis. For those who are susceptible, the condition is highly contagious. The best way to avoid onychomycosis is to keep feet clean and dry, washing them at least once a day and drying the toes well. Shoes and socks should be changed daily and should also be kept dry. The application of the compositions of the invention, particularly those lacking the usual fungicides, can be used as a means of preventing infection of the feet. The use of the sodium chlorite containing compositions in accord with the teachings disclosed herein can provide soothing and effective treatment of or prophylaxis against the conditions arising in at-risk individuals.

It has long been known that menthol, when applied to the nail can act as an antimicrobial used to combat infections that cause damage to the nails and other structures of the integument. It is now also known that menthol specifically inhibits the growth of a multitude of fungi known to cause onychomycosis (Phytother Res. 2003 Apr.; 17 (4):376-9). While camphor has little or no detectable anti-fungal activity, a composition of 4% Menthol with 2% Camphor is significantly more potent in its ability to kill toe nail fungus. It may be that the camphor acts as a penetration agent allowing better access of the menthol to the fungus under the toenail bed (International Patent No. WO/2002/022115 to McKenzie, et al., issued Mar. 21, 2002). However, when used in accord with the teachings of this disclosure, the antimicrobial activity is enhanced.

Thymol is known as a powerful antifungal. The essential oil of common thyme is made up of 20-55% thymol (Thymus Vulgaris. PDR for Herbal Medicine. Montvale, N.J.: Medical Economics Company. p. 1184). Thymol was used on bandages before the advent of antibiotics. (Thyme. A Modern Herbal. Hypertext version of the 1931 edition. Accessed: Dec. 14, 2006). Thymol has also been shown to be effective against the fungus that commonly infects toenails (Ramsewak R S, et al. In vitro antagonistic activity of monoterpenes and their mixtures against ‘toe nail fungus’ pathogens. Phytother Res. 2003 Apr.; 17 (4):376-9)

Third Planet Foods lists thymol as an antibacterial, antimicrobial and antifungal agent. Thymol is also used as a disinfectant, and is a popular ingredient in mouthwash. Many common medicines contain thymol. Medicines for acne, athletes foot, and hemorrhoids contain thymol, as well as ointments for chest congestion. Thymol is also a useful medication for treating hook worms and other parasites. The spread of E. coli and Staphylococcus aureus bacteria can be inhibited by the use of thymol.

The use of sodium chlorite at lower pH in the production of chlorine dioxide for disinfecting inanimate object, including fruits and vegetables is known. Sodium chlorite is used for industrial applications such as controlling microbial contamination in industrial cooling systems and towers. It is an oxidizer. Since it can destroy natural color matter without attacking the fibers themselves, it is used as a bleaching agent on textiles. When added to an acid base, it forms chlorine dioxide which is used in most municipal water supplies. Sodium chlorite, particularly when acidified, is currently being used as an ingredient in personal care products such as facial cleansing lotions, acne creams, mouthwash, toothpaste and contact lens preparations for its antimicrobial action and as a preservative. Ballinger has taught its use in generating chlorine dioxide for processing chickens. (U.S. Pat. No. 7,854,651) However, at pH ranges used in the methods of the invention under room temperature conditions the compositions are useful in treatment of the conditions as described herein.

It has previously been reported that ozonized olive oil can be used as an antifungal. Without relying on any particular theory, it is believed probable that ozonization of the oils as used in the invention results in production of peroxide, which has fungicidal/bacteriocidal properties. “Ozonated” plant and vegetable extracts have been researched in various countries, notably Cuba and Russia, where original research has been published for the last 40 years on the effects of ozonated sunflower and olive oils. Use of these ozonated oils offers good antimicrobial activity, show no tendency to result in micro-biological resistance, and show no harm to the patient or operator. When vegetable oils, which consist fundamentally of triglycerides are ozonised, ozonides, aldehydes, and peroxides are formed. Diaz reviewed past research from Cuba and Russia that showed these products are related with the observed biological effect of these oils.

In the pharmaceutical industry, ozonated olive and sunflower oils are recognized. The cosmetic industry lists ozonated olive, sunflower, jojoba and caster oils in the International Cosmetic Ingredient Dictionary and Handbook. “Ozonated olive oil must be kept refrigerated and is costly to produce because of the methods used to create it. It is believed that the introduction of sodium chlorite as described herein (which is a potent oxidizer) to oils such as olive oil results in benefits similar to or exceeding the benefits of the ozonation of OO. Sunflower oil is another substance well known to that release hydrogen peroxide when ozonized. These ozonized products may be used when the sodium chlorite acting as a carrier and penetrating agent, as well.

Any substance that releases peroxide when ozonized and is non-toxic should be useful for purposes of this invention. See 1) INTERNATIONAL JOURNAL OF AGRICULTURE &BIOLOGY, 1560-8530/2006/08-5-670-675, http://www.fspublishers.org, Antifungal Activity of Ozonized Olive Oil (Oleozone), NEVEEN S. I. GEWEELY, Department of Botany, Faculty of Science, Cairo University, Giza 12613, Egypt and 2. Ozonated Liquids in Dental Practice—A Review. Author: Dr Julian Holmes, Lime Technologies Holdings Ltd, Clinical Director. Date: April 2008. Ozonated Liquids in Dental Practice—A Review

### SUMMARY OF THE INVENTION

It is an object of the present invention to provide a method of topically treating the integument for damage arising from infections, especially, but not limited to, those caused by fungi. Such conditions may show themselves as blotches, atrophy, dermatitis, eczema, folliculitis, keratoses, melasmas; nodules, nummular dermatitis. The invention is exemplified using oil-containing compositions of the invention are soothing and improve appearance. The method is particularly useful for treating infections of the nail caused by dermatophytes However, other than oil-containing compositions may be used with the sodium chlorite to produce beneficial results. The invention may be practiced using a kit having at least two components, one containing sodium chlorite solution and one containing a suitable base, most preferably an oil, and/or a pharmaceutical antifungal agent, and/or a plant-based antifungal agent such as thymol and/or menthol. Other plant oils such as but not limited to tea tree, menthyl acetate, (derived from peppermint), oil of oregano and eucalyptus may be added to increase antimicrobial effects. Beeswax or other thickeners may be added. An anti-oxidant may be added to stabilize the composition. Also useful are some complex sugars or carbohydrates. The practice of concurrent invention can practiced using kits containing the compositions for use in the methods of the invention as described herein.

Preferred compositions of the invention are formulated using sodium chlorite at preferred concentrations of 0.2% to 3% (more preferably at 1.2-2.5%), with the most preferred range of 1.5% to 2%, and at a pH ranging from around 3 to 11, preferably above 6, more preferably around 10. It is preferred that the chlorite be stabile and not degrade significantly into chlorine dioxide, at least until treatment has begun. A very small amount (>10 ppm, preferably >1 ppm) of chlorine dioxide is acceptable prior to use and more generated during use is also acceptable. This can be achieved by raising the pH, not including oxidizing agents such as sodium hypochlorite, or adding buffers, antioxidants and other stabilizers. While there are other chlorite salts that may be used, such as but not limited to potassium, calcium, magnesium and other salts both organic and inorganic. Stabilizers commonly used in the pharmaceutical and cosmetic arts can be used in the compositions of the invention. For example, sodium bicarbonate can be used as a stabilizing agent as can lemon oil and or other citrus oils including grapefruit oil which can also be used as preservatives. When sodium chlorite is used in accord with the instructions in this application, formation of chlorine dioxide was not noted.

In small batches, 3.75 grams of sodium chlorite dissolved in 150 ml of water with sufficient glycolic acid to drop the pH to 6 is at this point a preferred embodiment. In another embodiment, 2% or 4 grams of sodium chlorite is dissolved in 150 ml of water and no glycolic acid is added. This produces a stable ph 9 or higher solution. Baking soda or other alkaline buffers may be added. No chlorine odor is associated with these compositions when they are stored in light-blocking tightly sealed containers.

In another method of this invention, a composition containing sodium chlorite and water can be formulated with addition of peroxide in the solution. It is also possible to use as solvents organic substances such as ethyl alcohol to which a compound such as polyvinyl pyrrolidone may added to provide compositions that create film. Polyethylene glycol may be used with or without pigments, which will tint the gel. Another component of the compositions used for such purposes is zinc oxide. Pigments used in the pharmaceutical and cosmetic arts may be used in the compositions of the invention.

### DETAILED DESCRIPTION OF THE INVENTION

Unless defined otherwise, all technical and scientific terms used herein have the same meaning as commonly understood by one of ordinary skill in the art to which the invention belongs. Although any methods and materials similar or equivalent to those described herein can be used in the practice or testing of the present invention, the preferred methods and materials are described herein. All references cited herein, including published or corresponding U.S. or foreign patent applications, issued U.S. or foreign patents, and any other references, are incorporated by reference in their entireties, including all data, tables, figures, and text presented in the cited references. Without relying on any theory, it is believed that when chlorite salts, preferably sodium chlorite and other components of the invention, such as olive oil and grapeseed oil, when combined may form peroxide. Without relying on any theory, it may be that the sodium chlorite facilitates penetration of the mixtures into the skin and nails. The products of the invention on formulation by mixing formed a rough emulsion. The application of the chlorite and some of the oils resulted in compositions, which lacked the objectionable smell often associated with products containing sodium chlorite. Some oils add odors that are less pleasant. Hence, choice of oil for any particular formulation could be chosen to avoid unpleasant odors, when practicable. Preservatives, and antioxidants, buffers, and odor modifiers used in the cosmetics and pharmaceutical arts can be used in compositions of the invention.

The invention may be practiced using nearly simultaneous administration of the chlorite and the other components used in the invention or may be practiced by mixing the components, then administering immediately to the target area. When administered separately, it is best to apply the solution first, then the oily components.

The chemical treatments of the present invention may be combined with other previously known medical treatments for fungal infections. Of particular interest is the use of laser or heat treatment of fungal infected nails sequentially with or as separate treatments, including alternating with the chemical treatments of the present invention.

Conventional pharmaceutical antifungal agents may be added separately to the area of the body being treated or they may be mixed with one of the components being applied. Concentrations and amounts similar to their known effective amounts are preferred.

The tolnaftate composition used in certain examples below is a water soluble tolnaftate composition MYCOCIDE: Active: Tolnaftate 1% inactive: Water, Caprylic/Capric Triglycerides, Isopropyl Palmitate, Polyquaternium 37, Propylene glycol. Dicaprylate Dicaprate, PPG-1 Trideceth-6, Cocamidopropyl Betaine, Cocamidopropylamine Oxide, Phemerol Chloride, Cetrimonium Chloride, Allantoin, Didecyldmonium Chloride, Quaternium-15.

The tolnaftate composition used in certain examples below is an oil soluble tolnaftate Active: Tolnaftate 1% (oil based) inactive: Sweet almond oil, Rice bran oil, Sesame seed oil, Safflower oil, Jojoba oil, Vitamin E Acetate, Tennox, Methyl Parben, Propyl Paraben, Paraben and Butyl Paraben.

Improvements in the appearance or fungal Nails is reflected in one or more of the following conditions:
Improvement of horizontal de-lamination results in the appearance of a unified nail.
Discoloration improves to a more natural color
Hard brittleness caused by fungus feels less rigid. Nails are easier to trim.
The texture of subungal keratosis softens from hard and cohesive to somewhat flaky.
After a few weeks use, built up subungal keratoses can be removed from beneath the nail with common nail tools, improving appearance.
   Effective treatment either cosmetically or actual treatment of fungal infection results in one or more of these improvements.

Sodium and other metal chlorite salts may function as penetration enhancers allowing a more effective transmission of other antimicrobial agent across the nail, skin or mucus membrane. Alternatively, the chlorite salt may act in conjunction with other known antimicrobial agents to enhance the total antimicrobial effect. A combination of these two effects may also be occurring. Either way, the net effect is greater than either treatment alone.

### Example 1

Two 2 drops of 2% sodium chlorite solution followed by 1 drop of 100% Olive oil was applied to nails evidencing symptoms of onychomycosis. The combination was tested on two subjects. Both subjects showed a reduction in fungal based nail discoloration within 2-10 days.

### Example 2

One drop of 2% Sodium chlorite solution was applied to each infected nail. Then an azole-containing composition was applied (clotrimazole and miconazole were tested). Lastly. 1 drop of olive oil was applied to each great toe and another drop of olive oil was distributed over the remaining 4 toes. The subject showed a reduction in nail discoloration. The nail appeared markedly clearer and the nail edge more defined. Additionally, dead cuticle was easily removed without use of nail tools after nine days of treatment.

### Example 3

One drop of 2% Sodium chlorite was applied to each infected nail. Then an azole containing composition was applied (miconazole was tested). Lastly 1 drop of tea tree oil was applied to each great toe and another drop of tea tree oil was distributed over the remaining 4 toes and to the skin on the heals. The subject showed a reduction in nail discoloration. The nail appeared markedly clearer and the nail edge more defined. Additionally, dead flaking skin on heels resolved to much smoother skin with markedly less flaking after twenty days of treatment.

### Example 4

A mixture of 2% sodium chlorite and olive oil was produced. Sodium chlorite was combined in a ratio of 1.5:1 sodium chlorite solution to olive oil and shaken. A rough emulsion was generated that did not separate when left undisturbed in room temperature for 9 hours. Some separation did occur after that time. The emulsion was applied to dry, dead cuticle on dry fingers. The cuticle was then pushed back. No tearing of healthy cuticle occurred. The cuticle pushed back easily and immediately as if it had been soaked for several minutes in water. Excess dead cuticle was easily removed without tearing healthy cuticle

### Example 5

Two 2 drops of 2% sodium chlorite and water solution followed by a pea sized dollop of a composition (consisting of approximately 77% grape seed oil, with 2% thymol, 2% menthol, 2% menthyl acetate and 17% beeswax and 0.01% tocopheryl (preservative) was applied to a nail that had KOH-tested positive for onychomycosis. The combination was tested on two subjects. Both subjects showed a reduction in fungal based nail discoloration within 2-10 days.

### Example 6

The composition featured in Example 5 was applied to infected nails once a day. Approximately 12 hours later, a composition containing 1% Tolnaftate with 2% sodium chlorite was applied to the infected toenail followed by the grapeseed oil based composition described in example 5. They were rubbed together. Alternating the treatments described herein, especially adding a specific pharmaceutical antifungal agent to the treatment regimen within a 24 hour period improves the effectiveness either composition when used alone.

### Example 7

Two 2 drops of 2% sodium chlorite and water solution followed by a pea sized dollop of a composition (consisting of approximately 77% grape seed oil, with 2% thymol, 2% menthol, 2% menthyl acetate and 17% beeswax and 0.01% tocopheryl) was applied to a deep crack near the knuckle of a diabetic. The skin was deeply fissured in a three point split roughly 0.75 cm in circumference and was characterized by raised swelling. Red could be seen in the depth of the fissure though no blood flowed from it. It had not healed for approximately 3 weeks, though emollients had been sporadically applied. Three treatments were applied over three days. Reduced swelling was observed within 2 hours of initial application. After 12 hours, the deep red in the fissure appeared pinker. The last two treatments were followed by a band-aid. Then, Subject abandoned all treatment. Surprisingly, 7 days later, lesion was healed.

### Example 8

Two 2 drops of 2% sodium chlorite and water solution followed by a pea sized dollop of a composition (consisting of approximately 77% grape seed oil, with 2% thymol, 2% menthol, and 17% beeswax and 0.01% tocopheryl) was used as a toothpaste. Tartar was judged significantly reduced by the attending dentist of subject.

### Example 9

Two 2 drops of 2% sodium chlorite and water solution followed by a pea sized dollop of a composition (consisting of glycerin and 2% thymol) was applied to a nail that evidenced discoloration characteristic of onychomycosis for three months. One year later, subject reports a clear nail with no relapse

### Example 10

A mixture of 2.07% sodium chlorite, tea tree oil and an azole (2% Miconazole) were rubbed onto the left heel for twenty days. The right heel remained untouched and served as a control. After twenty days skin was evaluated for softness, coarse roughened skin, and white deposits in the cracks of skin. The heel treated with composition was markedly softer and appeared healthier, with smoother appearance.

### Example 11

Several oils are used in place of olive oil. Plant and vegetable extracts include flax oil or linseed oil, safflower oil, hemp, evening primrose, safflower, chia, perilla, and grape seed oils/extracts, sunflower oil. However, sunflower oil gives off a rancid smell when oxidized. Other oils that may be used include walnut candle nut, soybean, corn, wheat germ, canola, sesame, cotton seed oil, rice bran, beechnut, sweet almond, avocado, theobroma, jojoba and castor oil. Some of these oils may give off a disagreeable odor. Most preferred are oils that readily release peroxides when oxidized in the presence of the chlorite. Additionally, other products such as herbal honey, may be used in place of the oils if they are capable of producing peroxide upon interaction with the sodium chlorite.

### Example 12

Example 5 was repeated with the same person using the composition above with added tolnaftate (1%). Other infected toes were treated only with the sodium chlorite and tolnaftate.

The tolnaftate and sodium chlorite had poor results with little change on a patient with difficult to treat long term nail fungus. However the addition of the oils composition provided in Example 5 resulted in superior results to tolnaftate and sodium chlorite alone as well as to the composition of Example 5.

### Example 13

The composition of Example 5 was used with the same composition without menthyl acetate. At no point were pharmaceutical antifungals used. At six months, one begins to see relapse. After 9 months, treatment with the new formulation with addition of menthyl acetate was begun. Immediate improvement was noted. After only 4 months of treatment with new menthyl acetate containing formulation, the pattern of general lightening of the dark yellow nail edge continues along with clear grow out.

### Example 14

2% Sodium chlorite and water solution was applied to infected toes. Immediately afterwards, benzoyl peroxide, to yield about 2% was mixed into a preformulated composition of 2.5% thymol, 2.5% menthol, 2.5% menthyl acetate, 17% beeswax with the remainder grapeseed oil. The mixture was applied to a nail that had tested KOH-positive for onychomycosis. The appearance of the nail significantly improved within 48 hours.

Discoloration in a jagged triangle shape had infiltrated about 70 mm into the nail past the white edge. After only 4 applications of this composition, the nail is pink.

### Example 15

In addition to the compositions mentioned above, the following 5 variations have been shown to be effective also. It should be apparent that slight differences in concentration and amount may also be used.

Variation A:

Composition 1-2% sodium chloride in water
(4 grams sodium chlorite in 150 ml water-small batch)

### Composition 2

6% thymol (6 grams small batch)
3% menthol (3 grams small batch)
Carrier: grapeseed oil (100 ml small batch)
0.24 oz beeswax small batch

Variation B

Composition 1-2% sodium chloride in water

### Composition 2

3% thymol (3 grams small batch)
3% menthol (3 grams small batch)
3% menthyl acetate (3 grams small batch)
Carrier: grapeseed oil (100 ml small batch)
0.24 oz beeswax small batch

Variation C

Composition 1-2% sodium chloride in water
Composition 2: good with azoles
3% menthol (3 grams small batch)
3% menthyl acetate (3 grams small batch)
Carrier: grapeseed oil (100 ml small batch)
0.24 oz beeswax small batch

Variation D

Composition 1-2% sodium chloride in water+1% baking soda

### Composition 2

3% thymol (3 grams small batch)
3% menthol (3 grams small batch)
3% menthyl acetate (3 grams small batch)
Carrier: grapeseed oil (100 ml small batch)
0.24 oz beeswax small batch

Variation E:

Composition 1-2% sodium chloride in water

### Composition 2

3% thymol (3 grams small batch)
Carrier: glycerin (100 ml small batch)
Thickener of your choice if desired.

These are representative of best compositions. Of the compositions described above, A and B have been tested the longest and show good cosmetic results without pharmaceutical antifungals.

1:1:1 ratios of herbal components are preferred. When mixing small batches, 2% thymol, 2% menthol, 2% menthyl acetate in 100 ml of grapeseed oil with 2.5 ounces of beeswax form an ointment that is not irritating to skin but improves nail appearance. Another preferred composition involves 2.66% thymol, menthol and menthyl acetate. Another effective composition does not entail use of menthyl acetate. Higher and lower ranges can be used.

When treatment is used as adjunct therapy with pharmaceutical antifungal agents to improve nail appearance, apply any pharmaceutical brush-on product to affected nails then mix sodium chlorite solution and ointment in a 1:2 ratio in hand and then apply mixture to affected nail. If the pharmaceutical antifungal is in lotion form mix the pharmaceutical containing lotion with sodium chlorite and ointment in a 1:1:2 ratio then apply to affected nail. Apply once or twice daily; up to 6 times in 24 hours.

Thymol is a known antifungal agent. Thymol can be used in combination with olive oil or in place of olive oil in a suitable base in accord with the teachings herein. A non-limiting list of agents similar to thymol may be used in place of thymol or in addition to thymol for beneficial effects include but are not limited to menthol, menthyl acetate, tea tree oil, eucalyptus oil, camphor, and phenol.

Other agents that may be used in the methods of the invention include eucalyptus crystals, lactic acid crystals, salicylic acid crystals and camphor crystals, thymol and menthol crystals. Additionally, oils derived from thymol or menthol may be deemed to be used in accord with the methods of the invention. Other beneficial oils, tinctures or crystals (where applicable) may be derived from the following non-limiting list Barberry, bitter almond, cedarwood, Rice bran, cinnamon, citronella, clove, coriander, eucalyptus, frankincense, Garlic, goldenseal, helicrysum, lavender, mustard, myrrh, Neem, Niaouli oregano, parsley, patchouli, Pau D'Arco, pennyroyal, red thyme, ravensara, rue, sage and tansy, Ides oil, Ribes Nigrum (black currant seed oil) may be used in the method of this invention. Additionally Oils or tinctures derived from California Bay (Umbellularia californica) Beebalm (Monarda didyma) Burdock (Arctium Lappa), especially the root thereof, Western Red Cedar (Thuja plicata) Sweet Root (Osmorhiza occidentalis), especially the root thereof, White Sage (Salvia apiana) California Soap Plant (Chlorogalum pomeridianum) Pacific Wax Myrtle (Myrica californica) California Mugwort (Artemisia vulgaris) Labrador Tea (Ledum glandulosum), Oregon Grape, especially the root thereof in all its forms, (Mahonia ssp.) (Mahonia aquifolia) (Mahonia nervosa). (Hydrastis canadensis), Oxeye Daisy (Chrysanthemum leucanthemum) may also be used in this invention. Essential oils, tinctures or crystals (where applicable) derived from plants containing D-LIMONENE are preferred including lemons, oranges, grapefruit, caraway, dill, bergamot, peppermint, spearmint and their derivatives therefrom such as menthyl acetate, a derivation from peppermint. Ingredients such as manuka oil, colloid silver and diatomaceous earth may be used in this invention. Such organically derived oils and ingredients may also be used in combination with the compositions exemplified herein to augment antifungal action or adjust fragrance.

The compositions are administered to dry nails. For example, in one instance the composition was applied to dry, dead cuticle on dry fingernails which had not been in water for ten hours or more. The cuticle was then pushed back. No tearing of healthy cuticle occurred. The cuticle pushed back easily and immediately as if it had been soaked for several minutes in water. Excess dead cuticle was easily removed without tearing healthy cuticle.

In another instance, the compositions used were applied to toenails that had long cuticles covering at least one third of the nail. After application, the dead over-long cuticle separated from healthy cuticle, leaving an aesthetically pleasing thin edge surrounding the nail. No tearing of healthy cuticle occurred. No mechanical implements such as cuticle scissors were used to achieve these beneficial effects.

In another instance, a split and cracked nail was aesthetically improved over a period of seven days. After the initial injury to the toenail, a thin white jagged split line was easily apparent to the casual observer. After seven days of treatment with compositions C and D (which are only distinguished by order of sequential application) upon moderately close inspection, the split was no longer easily observable though a small white edge could be observed on extremely close inspection.

Symptoms of itching and rash characteristic of tinea pedis between toes and on soles of feet ceased after three days application of compositions A, D, C and I. An early outbreak of Tinea Pedis symptoms ceased after two treatments with M1 and K.

When the treatment is applied topically it us best to cover the entire affected area. In the case of onychomycosis, the entire nail, under the nail and cuticle should be exposed to the compositions. Covering rough skin and nails requires more of the compositions than healthier nails. The compositions may be applied topically by usual means, including by dropper, spray or, in the case of the emulsion, by applicator. When applied to cracked skin or damaged nails it is advisable, though not necessary, to gently rub blended compounds(s) into the area to be treated.

Other compositions which combine a solution, cream, gel, or lotion consisting of around 0.005%-5%, preferably 3% peroxide, preferably but not limited to hydrogen peroxide and a sodium chlorite and water solution, cream gel or lotion at a ratio of 1 part peroxide to 2 parts sodium chlorite solution may have similar affects. At 1:1-1:8 ratios of peroxide to sodium chlorite solution benefit may be obtained. This composition can be used alone or with antibiotics or other beneficial agents to increase their penetration to aid treatment of bacterial and fungal infections of the skin and nails. A ph of around 3-12, preferably around 10, is preferred. A two step product or twin barrel dispenser may be used for administration of the chlorite and peroxide. Similar dispensers may be used with the sodium chlorite solution and oil or herbal honey or a plant derived antimicrobial(s) e.g. thymol in a containing base to form an emulsion at the time of simultaneous dispersal from the dispenser.

Another preferred method combines a solution, cream, gel, or lotion or two-part waxy “bead” consisting of any concentration of olive oil with compositions containing chlorite salts. Sodium chlorite as exemplified is preferred and economical. Preferred compositions contain sodium chlorite in any concentration, but is must preferred at between 0.75-3:0%, most preferably around 2%.

The products of the invention may be packaged containing sodium chlorite solution and other components packaged separately in kit form for with mixing and or simultaneous application at the time of use. The advantage over the use of the pre-mixed materials of the prior art is that, unlike the ozonized oils which need refrigerating, the components in the kit need not be refrigerated. Glycerin may be used in substitution for ozoniazable oil for many of the compositions described below.

As a suitable base, oil that can be ozonated with or without thickeners is preferred. However a liquid, cream, gel, paste, milk, or lotion which contains the usual pharmaceutical and cosmetic ingredients such as petroleum jelly, petrolatum, mineral oil, beeswax, glycerin, and/or propylene glycol may be used with an appropriate concentration of an antimicrobial. Preferably about 0.1-10%, most preferably around 1-6% thymol can be used. Menthol and/or menthyl acetate and/or other such agents popularly characterized as “essential” oils or products derived therefrom can be substituted for thymol or added to thymol; used individually or in combination.

When menthol and menthyl acetate are substituted for thymol according to this invention, effective ratios of menthol to menthyl acetate are 1:4, 3:7, 2:3, 1:1, 3:2, 7:3. Preferred ratios are between 2:3 and about 1:1-3:2. Most preferred ratios are 3:2. Though other ratios were tested, when thymol, menthol and menthyl acetate were used in combination, according to this invention, the most effective ratio was 1:1:1.

Compositions can also be formulated for use with other antibiotic or other active agents with sodium chlorite providing enhanced therapeutic response when the product is left on from 3 minutes to several hours.

Antifungals such as, but not limited to, Butoconazole, Clotrimazole, Econazole, Miconazole, Ketoconazole, Terconazole, Tioconazole, Lanoconazole and Luliconazole. bifonazole, clomidazole, croconazole, eberconazole, econazole, fenticonazole, Fluconazole, flutimazole, isoconazole, neticonazole, omoconazole, oxiconazole, setraconazole, sulconazole, fluconazole, itraconazole, Voriconazole, efinaconazole may be used in the method of the invention. Additionally, imadazole and azole class-antifungals sold over the counter in the USA can be layered with the products of the invention for enhancement of efficacy. A preferred imadazole antifungal of the present invention is Clotrimazole. It has antibiotic as well as antifungal properties. At present, Clotrimazole is used to treat yeast and fungal infections (including candidiasis and tinea) of the vagina, and skin such as athlete's foot, jock itch, and body ringworm. Another preferred imadazole antifungal agent is Miconazole nitrate, especially when used in conjunction with a gel or nail polish containing water and pentavitin.

Other agents that may effectively be used with the compositions of the present invention include terbinafine, Tababorole, natrifine, amorolfine, amphotericin B, nystatin, natamaycin, naftifine hydrochloride, flucytosine, Voriconazole, Luliconazole, griseofulvin, potassium iodide, butenafine, ciclopirox, ciloquinol (iodochlorhydroxyquin), haloprogin, tolnaftate, aluminum chloride, potassium permanganate, selenium sulphide, salicylic acid, zinc pyruthione, bromochlorsalicylanilide, methylrosaniline, tribromometacresol, undecylenic acid, polynoxylin, 2-(4-chlorphenoxy)-ethanol, chlorophensesin, ticlatone, sulbentine, ethyl hydroxybenzoate, dimazole, tolciclate, sulphacetamide, urea, benzoic acid and pharmaceutically acceptable salts thereof.

Alpha hydroxy acids are well known for use in the skin treatment and may be used with compositions of the present invention. Representative alpha hydroxy acids include mandelic acid, lactic acid, glycolic acid, etc. and salts thereof. One or more of these (or their salts) may also be used to serve as a pH adjusting agent or a buffering agent. While citric acid/citrate buffer is typically used, other pH-adjusting agents or buffering agents may be used alone or in combination with the alpha hydroxy acids of the present invention. Concentrations in the range of about 0.1% to about 20% are preferred. Depending upon the specific compound used and/or the intended frequency of use, narrower ranges are preferred. More preferred are ranges such as 0.2%-7% glycolic acid for daily use. Alpha hydroxyl acids, particularly Glycolic acids are particularly effective for cuticle treatments when combined with above 2% sodium chlorite.

It is also possible to provide the separate components (sodium chlorite composition and oil or similar components) in a multiple-barrel syringe or other multi-chambered container to co-extrude the different composition components which are either mixed immediately before applying. An alternative container may be a multipack containing individual dosages of one or more of the containers in, for example, capsules that may be punctured for administration to the target area. The components may also be held in the form of an emulsion with the active ingredients in one or more discontinuous phases in the emulsion. Water-in-oil emulsions are particularly preferred. This allows reactive components to be kept apart until mixed and applied.

While olive oil has been most extensively exemplified, other components such as grape seed oil, sunflower oil, safflower oil, or any of the other oils listed. Another component that may be used in addition or alone instead of oil is manuka honey. It is believed that any oil or product that can be easily ozonated may be used with the chlorite in the method of the invention. In some instances it might be beneficial to use an oil that has a disagreeable odor. For example, a product that gives an odor that is offensive to a pet may keep the animal from licking the treating mixture.

Though natural oils are preferred, the lipid phase of the topical compositions can be chosen from: mineral oils and mineral waxes; oils such as triglycerides of caprinic acid or caprylic acid and castor oil; oils or waxes and other natural or synthetic oils, esters of fatty acids with alcohols e.g. isopropanol, propylene glycol, glycerin or esters of fatty alcohols with carboxylic acids or fatty acids; alkylbenzoates; and/or silicone oils such as dimethylpolysiloxane, diethylpolysiloxane, diphenylpolysiloxane, cyclomethicones and mixtures thereof.

Half synthetic or modified oils are acceptable, (e.g. cocoglyceride, olive oil, sunflower oil, soybean oil, peanut oil, rape seed oil, sweet almond oil, palm oil, coconut oil, castor oil, hydrogenated castor oil, wheat oil, grape seed oil, macadamia nut oil and others). Acceptable oils include but are not limited to apolar oils such as linear and/or branched hydrocarbons and waxes e.g. mineral oils, vaseline (petrolatum); paraffins, squalane and squalene, polyolefines, hydrogenated polyisobutenes and isohexadecanes, dialkyl ethers such as dicaprylylether; linear or cyclic silicone oils such as cyclomethicones (octamethylcyclotetrasiloxane; Dimethicones including but not limited to PPG-2 and cetyldimethicone, hexamethylcyclotrisiloxane, polydimethylsiloxane, montoperones, poly(methylphenylsiloxane) and mixtures thereof.

Other fatty components suitable for use in the topical compositions of the present invention include polar oils such as lecithines and fatty acid triglycerides, namely triglycerol esters of saturated and/or unsaturated, straight or branched carboxylic acid. A non-limiting list of other fatty components that may be incorporated in topical compositions of the present invention are isoeikosane; neopentylglykoldiheptanoate; propyleneglykoldicaprylate/dicaprate; caprylic/capric/diglycerylsuccinate; butyleneglycol caprylat/caprat; C.sub.12-13-alkyllactate; di-C.sub.12-13 alkyltartrate; triisostearin; dipentaerythrityl hexacaprylat/hexacaprate; propylenglycolmonoisostearate; tricaprylin; dimethylisosorbid, the use of mixtures C.sub.12-15-alkylbenzoate and 2-ethylhexylisostearate, mixtures C.sub.12-15-alkylbenzoate and isotridecylisononanoate as well as mixtures of C.sub.12-15-alkylbenzoate, 2-ethylhexylisostearate and isotridecylisononanoate.

The oily phase of the compositions of the present invention can also contain natural vegetable or animal waxes such as bee wax, china wax, bumblebee wax and other waxes of insects as well as shea butter and cocoa butter.

Fatty substances which can be incorporated in the oil phase of the emulsion. Microemulsion, oleo gel, hydrodispersion or lipodispersion of the present invention can be chosen from esters of saturated and/or unsaturated, linear or branched alkyl carboxylic acids. Such esters include but are not limited to Isopropol Palmitate, octylpalmitate, octylcocoate, octylisostearate, octyldodecylmyristate, cetearylisononanoate, isopropylmyristate, isopropylpalmitate, isopropylstearate, isopropyloleate, n-butylstearate, n-hexyllaureate, n-decyloleat, isooctylstearate, isononylstearate, isononylisononanoate, 2-ethyl hexylpalmitate, 2-ethylhexyllaurate, 2-hexyldecylstearate, 2-octyldodecylpalmitate, stearylheptanoate, oleyloleate, oleylerucate, erucyloleate, erucylerucate, tridecylstearate, tridecyltrimellitate, as well as synthetic, half-synthetic or natural mixtures of such esters, for example jojoba oil.

Compositions in accordance with the invention can be in the form of a liquid, a lotion, a thickened lotion, a gel, a cream, a milk, an ointment, a paste, a powder, a tinted nail cosmetic make-up, a nail polish with brush applicator, or a solid tube stick and can be optionally be packaged as an aerosol and can be provided in the form of a mousse such as a aerosol mousse, a foam or a spray foams, sprays, sticks, a gel, a plaster, a powder, a cleanser, a soap or aerosols or wipes.

The compositions of the invention can also contain usual cosmetic or pharmaceutical additives, such as preservatives/antioxidants, fatty substances/oils, water, organic solvents, silicones, thickeners, softeners, emulsifiers, sunscreens, antifoaming agents, moisturizers, fragrances, surfactants, fillers, sequestering agents, anionic, cationic, nonionic or amphoteric polymers or mixtures thereof, propellants, acidifying or basifying agents, dyes, colorants, pigments or nanopigments, or any other ingredients usually formulated into cosmetics or medicaments.

A moisturizing agent may be incorporated into a topical composition of the present invention. Suitable humectants can be incorporated into a topical composition of the present invention such as glycerin, polypropylene glycol, polyethylene glycol, lactic acid, pyrrolidon carboxylic acid, urea, phopholipids, collagen, elastin, ceramides, lecithin sorbitol, PEG-4, and mixtures thereof. Additional suitable moisturizers are polymeric moisturizers of the family of water soluble and/or swellable/and/or with water gelating polysaccharides such as hyaluronic acid, chitosan and/or a fucose rich polysaccharide which is e.g. available as Fucogel® 1000 (CAS-Nr. 178463-23-5) by SOLABIA S.

The topical compositions of the present invention can contain the usual cosmetic or pharmaceutical additives such as but not limited to alcohols, including ethanol and/or isopropanol, cetanol, stearyl, low diols or polyols and their ethers, such as but not limited to polyoxyethylene cetyl ether; propyleneglycol, Dipropylene Glycol Dibenzoate, dicarylate dicaptrate, dicarylate dicaptrate, glycerin, ethyleneglycol, ethyleneglycol monoethyl- or monobutylether, propyleneglycol monomethyl- or -monoethyl- or -monobutylether, diethyleneglycol monomethyl- or monoethylether and analogue products, polymers, Capric Triglycerides, Isopropyl Palminate, Polyquaterternium 37, Dicaprylate Dicaprate, Phemerol chloride, Cetrimonium Chloride, (attracts proteins), Allantoin, Didecyldimonium Chloride, Quaternium-15, foam stabilisators; electrolytes and especially one or more thickeners. A non-limiting list of thickeners that may be used in formulations of the present invention to assist in making the consistency of a product suitable include cocamidopropyl betaine, cocamidopropylamine oxide, carbomer, siliciumdioxide, magnesium and/or aluminium silicates, beeswax, stearic acid, stearyl alcohol polysaccharides and their derivatives such as xanthan gum, hydroxypropyl cellulose, polyacrylamides, acrylate crosspolymers preferably a carbomer, such as Carbopole® of type 980, 981, 1382, 2984, 5984 alone or mixtures thereof.

Suitable neutralizing agents which may be included in the composition of the present invention to neutralize components such as e.g. an emulsifier or a foam builder/stabilizer.

Compositions in accordance with the invention can be in the form of a liquid, a lotion, a thickened lotion, a gel, a cream, a milk, an ointment, a paste, a powder, a make-up, or a solid tube stick and can optionally be packaged as an aerosol and can be provided in the form of a mousse such as a aerosol mousse, a foam or a spray foams, sprays, sticks, a gel, a plaster, a powder, a cleanser, a nail polish, a soap or aerosols or wipes.

Emulsifiers such as but not limited to sorbitan monostearate, cocamidopropyl betaine (organic from coconut oil) and solubizers may be used in the present invention.

The compositions of the invention can also contain usual cosmetic or pharmaceutical additives, such as preservatives/antioxidants, fatty substances/oils, water, organic solvents, silicones, thickeners, softeners, emulsifiers, sunscreens, antifoaming agents, moisturizers, fragrances, surfactants, fillers, sequestering agents, anionic, cationic, nonionic or amphoteric polymers or mixtures thereof, propellants, acidifying or basifying agents, dyes, colorants, pigments or nanopigments, or any other ingredients usually formulated into cosmetics including nail polish or medicaments.

The compositions of the present invention may also include the usual nail polish additives such as but not limited to ethyl acetate, butyl acetate, nitrocellulose, tribenzoin, propyl acetate, acetyl tributyl citrate, stearalkonium bentoinite, silica, HDI/Trimethhylol hexyllactone, panthenol, ascorbyl pamitate, silk powder, acyrlates copolymer, tetrabutyl phenyl hydroxybenzoate, alumina, acrylate copolymer/styrene acrylate copolymer propylene glycol n-butyl ether, and colorants. A water-based polish is preferred.

Though Vitamin E Acetate is preferred, other antioxidants such as but not limited to Tetrabutyl phenyl hydroxybenzoate may be used according to this invention.

Preservatives such as but not limited to, propylparaben, Sodium propyl p-hydroxybenzoate, butylated hydroxytoulene msds and sigma and/or butylated hydroxyanisole msds, Methylparaben, Butyl Paraben and/or Propyl paraben may be used. Tennox may be used.

Though sodium chlorite is exemplified and preferred, any nail penetrator can theoretically be used according to this invention such as but not limited to DMSO.

Treatments described herein promote healthy disease-free nail growth but do not rely on nail growth as a measure of positive results. Even portions of the nail that are discolored and distorted by the presence of fungus return to an aesthetically pleasing normal healthy color and texture when the treatments described herein are applied.

Fungus Quality

1—Fungus is reduced, is easily removed, nail flattens.

2—Improvement

3—No Change

4—Relapse. Formerly flakey, easy-to-remove fungus transforms back into a unified, hard, difficult to penetrate plastic-like wall.

Skin Roughness—Reflects Compound's Effect on Fungus Impregnated Skin Around the Nails and Cuticles.

1—Improvement

2—No Change

3—Slight Skin Roughness Observed

4—Severe Skin Roughness is Observed

Healthy Nail Appearance

1—Dramatic improvement

2—Improvement

3—No change

4—Relapse. The white edge of the nail widens. Lateral striations originating at the nail edge begin to intrude on the body of the nail.

Irritation of Skin and Nail

1—Feel nothing

2—Slight pleasant tingle

3—Discomfort

Whenever Stage 4 occurs, or an irritation level 3 is reached, testing of listed Composition was abandoned.

TABLE 1
Except for Composition Q, which was tested on a lightly infected
fingernail, all compositions were tested on thickened discolored
toenails indicating presence of a bacterial or fungal infection.
Subjects L and J2 had received positive KOH cultures before treatment.
All subjects had been diagnosed with onychomycosis by a podiatrist
or dermatologist prior to beginning the study. Infections ranged
from light (only distal portion of nail affected) to severe (entire
nail affected). Only Subject S had lightly infected nails. Subject
k had moderately infected nails. All others were severe.

| | Fungus Quality | Skin Roughness | Nail Appearance | Irritation |
|---|---|---|---|---|
| A. | 1 | 2 | 1 | 1-2 |
| B. | 4 | 4 | 4 | 1 |
| C. | 1 | 1 | 1 | 1-2 |
| D. | 1 | 1 | 1-2 | 1-2 |
| E. | 1 | 1 | 2-1 | 1 |
| F. | 1 | 1 | 2-1 | 1 |
| G. | 3-4 | 2 | 4 | 1 |
| G1. | 1 | 1 | 1 | 1 |
| H. | 3 | 2-1 | 3 | 1 |
| H1. | 2 | 1 | 2-3 | 1 |
| I. | 2 | 1 | 2 | 1 |
| J. | 1 | 1 | 1.5 | 1 |
| K. | 1-2 | 1 | 1-2 | 1 |
| L. | 2 | 1 | 2 | 1 |
| M. | 1-2 | 1 | 2 | 1 |
| M1. | 1 | 1 | 1 | 1 |
| N. | 1 | 1 | 1-2 | 1 |
| O. | 1 | 1 | 1-2 | 1 |
| P. | 1 | 1 | 1 | 1 |
| Q*. | 1 | 1 | 1 | 2 |
| R. | 1 | 1 | 1 | 1-2 |
| R1. | 1 | 1 | 1 | 1-2 |
| S. | 1 | 1 | 1 | 2 |
| S1. | 2-1 | 2 | 2 | 2 |
| T. | 2-3 | 2-1 | 2-3 | 2 |
| U. | 1-2 | 1 | 1 | 1-2 |
| V. | 1-2 | 1 | 1 | 1-2 |
| W. | 2 | 1 | 2-3 | 2 |
| X | 2-1 | 1-2 | 2 | 2 |
| Y. | 2-1 | 2-1 | 4(discoloration) | 2 |
| Z. | 2-3 | 2-1 | 3 | 1 |
| Za. | 2 | 1 | 2 | 1 |
| Zb. | 2-1 | 1 | 2-1 | 1 |
| Zc. | 1-2 | 1 | 1-2 | 1 |
| Zd. | 1-2 | 1 | 1 | 2 |
| Ze. | 1 | 2 | 1 | 1 |
| Zf. | 1-2 | 4 | 3 | 2-3 |
| Zg. | 1 | 1 | 1 | 1 |
| 1. | 1 | 1 | 1 | 1-2 |
| 1a. | 1 | 1 | 1 | 1-2 |
| 1b. | 1 | 1 | 1+ | 1-2 |
| 2. | 1 | 1 | 1 | 1-2 |
| 2a. | 1 | 1 | 1 | 1-2 |
| 2b. | 1 | 1 | 1+ | 1-2 |
| 3. | 3 | 1 | 3 | 1-2 |
| 4. | 3 | 1 | 3 | 1-2 |
| 5. | 3 | 1 | 3 | 1-2 |
| 6. | 3 | 1 | 3 | 1-2 |
| 7. | 2 | 3 | 2 | 1-2 |
| 8. | 2 | 1 | 2 | 1-2 |
| 9. | 1-2 | 1 | 1-2 | 1-2 |
| 10. | 1 | 1 | 1 | 1 |
| 11. | 3-2 | 1 | 3-2 | 1 |
| 12. | 1-2 | 1-2 | 1-2 | 1 |
| 13. | 3-2 | 1 | 3-2 | 1 |
| 14. | 2 | 1-2 | 2 | 1 |
| 15. | 1-2 | 1-2 | 1-2 | 1 |
| 16. | 1 | 1-2 | 1 | 1 |
| 17. | 1 | 1-2 | 1 | 1 |
| 18. | 2-3 | 1-2 | 3-4 | 1 |
| 19. | 2-3 | 1 | 2-3 | 1 |
| 20. | 1 | 1 | 1-2 | 1 |

Q* tested on fingernail.

11. 2% sodium chlorite and 1% water based Tolnaftate composition. Substances were mixed then applied.

12. 2% sodium chlorite, 1% water-based Tolnaftate, 2% Menthyl acetate, 2% Menthol, BHT, 100 ml. Grapeseed oil, 2.5 oz. Beeswax.

13. 1% oil based Tolnaftate, Sweet almond oil, BHT, Sunflower oil, Peppermint oil, Seame seed oil, Vitamin E, 2% Menthol, BHT, 100 ml., Sesame seed oil, Alpha Tocopheryl

13a. 1% oil based Tolnaftate, Sweet almond oil, BHT, Sunflower oil, Peppermint oil, Seame seed oil, Vitamin E, 2% Menthol, BHT, 100 ml. Grapeseed oil, Sesame seed oil, 2% Thymol, 2.50 Menthol, 2.50 Menthyl acetate, 2.3 oz Beeswax

14. 2% sodium chlorite, 1% oil based Tolnaftate, Sweet almond oil, BHT, Sunflower oil, Peppermint oil, Seame seed oil, Vitamin E, 2% Menthol, BHT, 100 ml., Sesame seed oil, Tolnaftate Acetate

15. 2% sodium chlorite, 1% oil based Tolnaftate, Sweet almond oil, BHT, Sunflower oil, Peppermint oil, Seame seed oil, Vitamin E, 2% Menthol, BHT, 100 ml. Grapeseed oil, Sesame seed oil, 2% Thymol, 2.50 Menthol, 2.50 Menthyl acetate, 2.3 oz Beeswax

16. 2% sodium chlorite. 1% oil based Tolnaftate, 2% Menthyl acetate, 2% Menthol, BHT, 100 ml. Grapeseed oil, 2.5 oz. Beeswax. Trace Tocopheryl acetate

17. 1% oil based Tolnaftate, 2% Menthyl acetate, 2% Menthol, BHT, 100 ml. Grapeseed oil, Sesame seed oil, 2% Thymol, 2.5% beeswax

18. Tolnaftate 1% (oil based) Sweet almond oil, Rice bran oil, Sesame seed oil, Safflower oil, Jojoba oil, Vitamin E Acetate, Tennox, Methyl Parben, Propyl Paraben, Paraben and Butyl Paraben.

19. 2% Sodium chlorite, Tolnaftate 1% (oil based) Sweet almond oil, Rice bran oil, Sesame seed oil, Safflower oil, Jojoba oil, Vitamin E Acetate, Tennox, Methyl Parben, Propyl Paraben, Paraben and Butyl Paraben.

20. 2% Sodium chlorite, Tolnaftate 1% (oil based) Sweet almond oil, Rice bran oil, Sesame seed oil, Safflower oil, Jojoba oil, Vitamin E Acetate, Tennox, Methyl Parben, Propyl Paraben, Paraben and Butyl Paraben, 2% Menthyl acetate, 2% Menthol, BHT, 100 ml. Grapeseed oil, 2.5 oz. Beeswax.

### Compositions Defined

A. A drop of a 2% Sodium chlorite in water solution was dropped onto each infected toe or portion of the foot that required care. Next an antifungal (miconazole 2%) was applied to same. Next tea tree oil was rubbed in or applied to same.

B. one to two drops of 2.07% sodium chlorite in water solution was dropped onto each infected toe. This was combined with Tea Tree oil.

C. one to two drops of 2.07% sodium chlorite in water solution was applied to each infected toe. Next an antifungal (2% Clotrimazole) was applied. Next 100% 1 drop of olive oil was applied and dispersed over the great toe. Another drop was spread over other 4 toes. Leftover on hand was swiped over the left heel.

D. One drop of olive oil was applied to each toe. Next an azole-based antifungal was applied (2% Clotrimazole). Next, 1 drop of a 2.07% sodium chlorite (SC) in water solution was dropped onto each infected toe and massaged in until one could not see the white antifungal lotion.

E. One drop of 2.0% sodium chlorite in water solution was dropped onto each infected toe. This was combined with 1 drop of 100% olive oil on the toenail and rubbed into toenail briefly.

F. A solution containing 2% sodium chlorite and water was combined with 100% olive oil in a ratio of 1.5 parts sodium chlorite solution to 1 part olive oil. Upon shaking, bubbles appeared then resolved into what appeared to an emulsion. The resulting substance was applied to each infected toe.

G. A 1.5% thymol alone in glycerin (1.5% T+G): A solution containing 1.5% heat liquefied thymol crystals was prepared in 100% glycerin. The resulting substance was applied to each infected toe.

G1 A 1.5% sodium chlorite in water solution was applied to affected nail; then Composition G was applied.

H. A composition of 2% thymol in glycerin (2% T+G) was prepared. A solution containing 1.5% heat liquefied Thymol crystals were combined with glycerin to make 100%. The resulting substance was applied to each infected toe.

H1. A 2% sodium chlorite and water solution was applied to affected nail, followed by application of Composition G.

I. (2% T+GS) A composition containing 2% heat liquefied thymol crystals combined with 100% grape seed oil was prepared. The resulting substance was applied to each infected toe after application of 2% sodium chlorite in water solution had been applied by dropper to each affected toe.

J. (T+M+GS) A composition containing 2% menthol crystals liquefied in grape seed oil was combined with 2% heat-liquefied thymol crystals in grape seed oil. The resulting substance was applied to each infected toe after application of 2% sodium chlorite in water solution by dropper was applied to the affected toes.

K. (T+GS+Beeswax) A composition containing 2% heat liquefied thymol crystals was combined with grape seed oil then melted in beeswax (0.25 oz beeswax in 3 oz of tymol in grapeseed oil). The resulting substance was applied to each infected toe after application of 2% sodium chlorite and water solution by dropper to each affected toe.

L. (M+GS+Beeswax) A composition containing 2% menthol crystals liquefied in grape seed oil, then melted in beeswax (0.25 oz beeswax in 3 oz of M in grapeseed oil). The resulting substance was applied to each infected toe after application of 2% sodium chlorite and water solution by dropper.

M. (T+M+GS+Beeswax) A composition containing 2% menthol crystals liquefied in grape seed oil was combined with 2% heat liquefied thymol crystals in grape seed oil, then melted in beeswax (0.25 oz beeswax in 100 ml of thymol in grapeseed oil). The resulting substance was applied to each infected toe.

M1. A 2% sodium chlorite and water solution was applied to each infected toe followed by Composition M.

Olive Oil/Thymol/Menthol

N. (T+OO) A composition containing 2% heat liquefied thymol crystals combined with olive oil was prepared. The resulting composition was applied to each infected toe after application of 2% sodium chlorite and water solution by dropper to each affected toe.

O. (T+M+OO) A solution containing 2% menthol crystals liquefied in olive oil was combined with 2% heat-liquefied thymol crystals in olive oil. The resulting composition was applied to each infected toe after application of 2% sodium chlorite and water solution to each affected toe.

P. 1.5% thymol and 1.5% menthol liquefied in canola oil was applied to each affected toe after application of 1.5% sodium chlorite and water solution by dropper.

Q. Thymol crystals are combined with an extender that does not adulterate the chemical properties of the thymol (such as but not limited to corn starch) to make a mixture. The resulting mixture is lightly packed under the affected nail.

Q1. A 1-2% sodium chlorite in water is added before or after Method Q.

R. Thymol crystals are combined with an extender that does not adulterate the chemical properties of the thymol (such as, but not limited to, corn starch). The resulting mixture is lightly packed under each affected nail. Next, Composition M is smoothed over the entire nail, periungal tissue and cuticle. In the process, M is glided over the dry thymol mixture to act as a seal. Compositions M1, K and L (without SC) can be substituted for M. Method R can be alternated with Compositions K or L; for example: use method R in the morning and method M1 in the evening.

R1. A 1-2% sodium chlorite in water solution is applied by dropper before or after Method R.

S. A 2% sodium chlorite solution was applied to affected nail followed by 5% vinegar solution in water applied by dropper. Composition M was then applied.

S1. A 2% sodium chlorite was applied to an affected nail followed by 5% acetic acid solution.

T. A 2% sodium chlorite was applied by dropper followed by 2% Thymol heat-liquefied in mineral oil is applied by dropper to a fingernail.

U. A solution of 2% sodium chlorite was applied by dropper followed by 100% raw unrefined “active+15” Manuka honey applied to nail.

V. A solution of 2% sodium chlorite was applied by dropper followed by Manuka honey which was mixed with Composition M before use and stored in container. Ratio was 1:1. Manuka honey:Composition M.

W. A solution of 2% sodium chlorite was applied by dropper followed by a composition as described: Ingredients according to M4 were prepared and cooled. During “cool down” the mixture was continually stirred. When temperature had dropped to around 97 degrees, Manuka honey was added. Ratio 1:1; M4 composition: 1 Manuka honey: 1. Also during cool down a solution containing 0.04 g of (5,746 IU) d-alpha tocopheryl acetate was blended into the mixture.

X. Composition M was prepared. During cool down a solution containing 0.04 g of (5,746 IU) d-alpha tocopheryl acetate was blended into the mixture, stored, then applied to nail after application of 2% sodium chlorite.

Xa 0.01 g of (5,746 IU) d-alpha tocopheryl acetate was substituted for 0.04 g of same.

Y. Composition M was prepared. During cool down a solution containing 2% rosemary oleoresin was blended into the composition. Ratio 1 ml Rosemary Oleoresin:100 ml Composition M.

Z. A 2% sodium chlorite solution was first applied by dropper followed by a solution containing 2% heat liquefied Thymol and 5% acetic acid solution in water and applied to nail.

Za. A 2% sodium chlorite solution was applied by dropper and was followed by application of coconut oil.

Zb. A 2% sodium chlorite solution is applied by dropper followed by coconut oil and 3% heat liquefied menthol.

Zc. A 2% sodium chlorite solution is applied by dropper followed by coconut oil and 3% heat liquefied thymol and 2% menthol in beeswax.

Zd. Composition Zc followed by application of vinegar solution which contains 5% acetic acid is applied between the use of sodium chlorite by dropper and the coconut oil beeswax/carnauba blend.

Ze. A 2% sodium chlorite solution is applied by dropper to infected nail (designated Part 1A). Next, the following composition (designated Part 1B) is preferably brushed or otherwise applied to the nail, thinly coating its entire surface.

Part 1B contains a composition comprised of 30% Ethyl alcohol in water which is then is combined in a beaker. The beaker is placed on a magnetic mixer. A 9% polyvinyl pyrrolidone and 5% Polyethylene glycol is added gradually at 200 r/min and temperature is raised to about 40 degrees C. Around 3% ground peroxide is mixed thoroughly into composition after polyethylene glycol and polyvinyl pyrrolidone completely dissolves. Cool down.

Zf. Composition Ze is used as described above except that in Part 1B, Six (6)% peroxide was used. Two applications were applied within 30 minutes of one another.

Zg. Composition Zf was used as described above except that Part 1b was combined with Composition J at a 1:1 ratio. The composition was applied one time in a period of 24 hours.

1. (T+M+MA+GS+Beeswax) A solution containing 2% menthol crystals and 2% menthyl acetate dissolved in 100 ml of grape seed oil was then combined with 2% heat liquefied thymol crystals, then mixed with melted 0.57 oz. beeswax. 0.01% Tocopheryl acetate was added. The resulting substance was applied to each infected toenail after application of 2% sodium chlorite solution.

1a. (T+M+MA+GS+Beeswax and Tolnaftate) A solution containing 2% menthol crystals and 2% menthyl acetate dissolved in 100 ml of grape seed oil was then combined with 2% heat liquefied thymol crystals, then mixed with melted 0.57 oz. beeswax. 0.01% Tocopheryl acetate was added. The resulting substance was applied to each infected toenail after application of 2% sodium chlorite solution with 1% Tolnaftate.

1b. Preferably apply compositions (1) and (1a) twice a day. Alternate the applications of (1) and (1a) over a twenty-four hour period.

2. (M+MA+GS+Beeswax) A solution containing 2% menthol crystals dissolved in 2% menthyl acetate and 100 ml of grape seed oil and then mixed with melted 0.57 oz. beeswax. 01% Tocopheryl acetate was added. The resulting substance was applied to each infected toenail after application of 2% sodium chlorite solution.

2a. (M+MA+GS+Beeswax) A solution containing 2% menthol crystals dissolved in 2% menthyl acetate and 100 ml of grape seed oil and then mixed with melted 0.57 oz. beeswax. 01% Tocopheryl acetate was added. The resulting substance was applied to each infected toenail after application of 2% sodium chlorite solution. A light coating of 2% clotrimazole was applied immediately afterward.

2b. (M+MA+GS+Beeswax) A solution containing 2% menthol crystals dissolved in 2% menthyl acetate and 100 ml of grape seed oil and then mixed with melted 0.57 oz. beeswax. 01% Tocopheryl acetate was added. The resulting substance was applied to each infected toenail after application of 2% sodium chlorite solution. 1% Tolnaftate was then applied to the nail in the form of Mycocide NS.

3. (T+M+MA+GS+Beeswax) A solution containing 2% menthol crystals liquefied in 4% menthyl acetate and 100 ml of grape seed oil was combined with 2% heat liquefied thymol crystals, then mixed with melted 0.56 oz. beeswax. 0.01% Tocopheryl acetate was added. The resulting substance was applied to each infected toenail after application of 2% sodium chlorite solution.

4. (T+M+MA+GS+Beeswax) A solution containing 3% menthol crystals was dissolved in 3% menthyl acetate and 100 ml of grape seed oil and then combined with 2% heat liquefied thymol crystals, then mixed with melted 0.6 oz. beeswax. 0.01% Tocopheryl acetate was added. The resulting substance was applied to each infected toenail after application of 2% sodium chlorite solution.

5. (T+M+MA+GS+Beeswax) A solution containing 2% menthol crystals dissolved in 4% menthyl acetate and 100 ml of grape seed oil was then combined with 2% heat liquefied thymol crystals, then mixed with melted 0.57 oz. beeswax. 0.01% Tocopheryl acetate was added. The resulting substance was applied to each infected toenail after application of 2% sodium chlorite solution.

6. (T+M+MA+GS+Beeswax) A solution containing 2.70 g menthol crystals liquefied in 1.80 g menthyl acetate and 100 ml of grape seed oil was left overnight then combined with 2.70 g heat liquefied thymol crystals, then mixed with melted 0.57 oz. beeswax. 0.01% Tocopheryl acetate was added. The resulting substance was applied to each infected toenail after application of 2% sodium chlorite solution.

7. (T+MA+glycerin) A solution containing 1 g thymol crystals was liquefied in 1 g menthyl acetate and combined with 50 ml of glycerin. The resulting substance was applied to each infected toenail after application of 2% sodium chlorite solution.

8. (T+MA+GS+Beeswax) A solution containing 1 g thymol crystals was liquefied in 1 g menthyl acetate in 50 ml of grape seed oil, then combined with 0.30 oz. melted beeswax. 01% Tocopheryl acetate was added. The resulting substance was applied to each infected toenail after application of 2% sodium chlorite solution.

9. (M+MA+GS+Beeswax) A solution containing 3% menthol crystals dissolved in 2% menthyl acetate and grape seed oil and then mixed with melted 0.57 oz. beeswax. 01% Tocopheryl acetate was added. The resulting substance was applied to each infected toenail after application of 2% sodium chlorite solution.

10. (M+MA+glycerin) A solution containing 3% menthol crystals dissolved in 2% menthyl acetate and glycerin. The resulting substance was applied to each infected toenail after application of 2% sodium chlorite solution.

\11. 2% sodium chlorite and 1% water based Tolnaftate composition. Substances were mixed then applied.

12. 2% sodium chlorite, 1% water-based Tolnaftate, 2% Menthyl acetate, 2% Menthol, BHT, 100 ml. Grapeseed oil, 2.5 oz. Beeswax.

13. 1% oil based Tolnaftate, Sweet almond oil, BHT, Sunflower oil, Peppermint oil, Seame seed oil, Vitamin E, 2% Menthol, BHT, 100 ml., Sesame seed oil, Alpha Tocopheryl

13a. 1% oil based Tolnaftate, Sweet almond oil, BHT, Sunflower oil, Peppermint oil, Seame seed oil, Vitamin E, 2% Menthol, BHT, 100 ml. Grapeseed oil, Sesame seed oil, 2% Thymol, 2.50 Menthol, 2.50 Menthyl acetate, 2.3 oz Beeswax

14. 2% sodium chlorite, 1% oil based Tolnaftate, Sweet almond oil, BHT, Sunflower oil, Peppermint oil, Seame seed oil, Vitamin E, 2% Menthol, BHT, 100 ml., Sesame seed oil, Tolnaftate Acetate

15. 2% sodium chlorite, 1% oil based Tolnaftate, Sweet almond oil, BHT, Sunflower oil, Peppermint oil, Seame seed oil, Vitamin E, 2% Menthol, BHT, 100 ml. Grapeseed oil, Sesame seed oil, 2% Thymol, 2.50 Menthol, 2.50 Menthyl acetate, 2.3 oz Beeswax

16. 2% sodium chlorite. 1% oil based Tolnaftate, 2% Menthyl acetate, 2% Menthol, BHT, 100 ml. Grapeseed oil, 2.5 oz. Beeswax. Trace Tocopheryl acetate

17. 1% oil based Tolnaftate, 2% Menthyl acetate, 2% Menthol, BHT, 100 ml. Grapeseed oil, Sesame seed oil, 2% Thymol, 2.5% beeswax

18. Tolnaftate 1% (oil based) Sweet almond oil, Rice bran oil, Sesame seed oil, Safflower oil, Jojoba oil, Vitamin E Acetate, Tennox, Methyl Parben, Propyl Paraben, Paraben and Butyl Paraben.

19. 2% Sodium chlorite, Tolnaftate 1% (oil based) Sweet almond oil, Rice bran oil, Sesame seed oil, Safflower oil, Jojoba oil, Vitamin E Acetate, Tennox, Methyl Parben, Propyl Paraben, Paraben and Butyl Paraben.

20. 2% Sodium chlorite, Tolnaftate 1% (oil based) Sweet almond oil, Rice bran oil, Sesame seed oil, Safflower oil, Jojoba oil, Vitamin E Acetate, Tennox, Methyl Parben, Propyl Paraben, Paraben and Butyl Paraben, 2% Menthyl acetate, 2% Menthol, BHT, 100 ml. Grapeseed oil, 2.5 oz. Beeswax.

Carnauba wax, shea butter or petroleates can be added to beewax in compositions or substituted for beeswax in compositions of the invention. Cosmetic grade Colorants may be added to mixtures. Petroleum jelly may be substituted for beeswax. Other oils may be substituted for grapeseed oil in Compositions K, L, M, M1. Most preferred are oils that readily release peroxides when oxidized in the presence of the chlorite. Olive oil and sunflower oil are examples of oils that can be substituted. (The listing of oils for use in the methods of the invention is provided for exemplification, and not as limitations, since other oils used for cosmetics may be used in replacement for the oils identified by name.)

Subjects were instructed to apply the compositions as directed twice a day. Compliance varied from stringent to moderate. One subject only applied compositions once a day and still showed steady improvement.

Subject T was given 3 “phases” of treatment, consisting of more than 1 month and not less than 9 months of treatment with Composition M1, followed by 1 month and not less than 9 months of treatment with Composition 2a, consisting of more than 1 month and not less than 9 months of treatment with Composition 1. Ideally treatment intervals of 3-5 months are preferred before substitution with another composition. Without being bound to a particular theory, it is believed that changing compositions in this manner interferes with adaption by nail fungus. At each composition change, dramatic improvement in nail appearance was observed.

Subjects identified herein as L and J3 have extremely fungus-compromised nails characterized by a raised nails, discoloration and, in the case of J3, pain. Both men were treated over the course of at least 34 days with sodium chlorite and water solution and an azole based composition. At that time, different combinations of thymol and menthol based compositions and sodium chlorite and water solution were tested on subjects' nails, recorded, photographed and analyzed. A combination of thymol in glycerin and thymol alone were the poorest performers against nail fungus as compared to progress with sodium chlorite in water solutions given in conjunction with an azole, with thymol in glycerin alone having no measureable effect and thymol in glycerin after application of sodium chlorite and water solution a more subtle positive effect.

In a third subject's lightly infected nail, thymol in glycerin prepped with a drop of 2% sodium chlorite in water solution performed well enough to clear the nail within 17 days. At that point, sodium chlorite in water solution was discontinued and thymol in glycerin alone was used for 7 days. The nail began to darken indicating relapse. Sodium chlorite in water solution was again included in the treatment plan. After 14 days, the nail appears clear again. Hence, it appears that sodium chlorite in water solution given in conjunction with the thymol compositions improves response against nail fungus. Further, when sodium chlorite in water solution is used in conjunction with thymol and/or menthol in an oil base, with and without beeswax, notable effects were perceived within 1 week. One year later, nail is reported clear with no relapse.

A fourth subject uses Composition X1 to left foot for ten months. M1 is applied for three months, and then switched to X1 for the following 7 months because both M1 and X1 appear to give similar results and an antioxidant is desirable. Recovery continued at the proximal edge (after repeated instruction to apply medication at cuticle was comprehended) but early whole-nail aesthetic improvement plateaued. At ten months, Composition 1 and composition 2 are applied to the right and left foot respectively. After 11 days, the entire nail of each infected toenail of both feet rapidly improves in the appearance, with the right foot (composition 1) showing slightly superior improvement over composition 2.

The addition of beeswax improved all oil based compositions with which it was tested (Composition M1 exemplified.)

The results using 1-2% sodium chlorite solution with the oil-based compositions compared favorably with the result of 2% sodium chlorite and azole-based antifungals. It is often beneficial to avoid use of azoles because many people are allergic to azoles. Contrary to azoles, both thymol and menthol are recognized for their low toxicity and are naturally derived products.

A comparison was performed of the comparative effectiveness of several compositions containing the same quantities of sodium chlorite, beeswax, and grapeseed oil. The distinction in the compositions was that:

Comp M1 contains 2% thymol and 2% menthol;

Comp K contains 2% thymol; and

Composition L contains 2% menthol.

Superior results were achieved with composition M1, followed by K and L in that order. That Composition M which contains 2% thymol and 2% menthol in an oxidizable oil (in this case grapeseed oil) combined with beeswax was surprisingly effective without sodium chlorite was interesting. However, Composition M it was less effective than Composition M1 and K, and slightly less effective than Composition L which also was used with sodium chlorite.

Other surprising benefits of the treatments described herein include the following:

1) There was notable cosmetic improvement of the appearance of the nail cuticle and surrounding skin with compositions as noted in data chart. No cuticle scissors were used and dead rough skin resolved within one week with oil or oil and beeswax-based compositions.

2) After 5 days of treatment with sodium chlorite solution and an azole (Compositions including both 1 and 2% Clotrimazole and a solution comprised of water and 2% sodium chlorite), Subject J reported cessation of pain in his toes.

3) Subject L had three prominent corns on his toes. Within one week after beginning treatment with Composition S (2% Sc and 2% Clotrimazole), 3 corns of varying intensity began to reduce in size and pain. By day 21, one minor corn was gone, a second almost completely gone, and a third greatly reduced. As treatment with the sodium chlorite and thymol and/or menthol compositions began and progressed, the reduction of the most prominent corn (which was surmounted by a white hardened pimple atop raised reddened skin) continued to regress rapidly over the next two weeks. The white hardened pimple is gone and the skin is only slightly reddened. Pronounced callusing is reduced. Treatment was abandoned and one corn returned but the (two other corns did not return. Patient whose corn had been reduced by his podiatrist was told the corn would come back. When the pimple began to form again, treatment was applied again. Corn reduced in size and the pimple disappeared. Apparently sodium chlorite and azoles or sodium chlorite and thymol and/or menthol reduce and/or can eliminate symptoms from corns. Sodium chlorite is a known anti-inflammatory. This result surpasses the work of previously used anti-inflammatory agents. Reduction of corns is significant.

Twice daily treatment with Composition M1 followed by Composition K was effective at softening a plantar wart. The black center of wart is gone or lightened and heavy callused skin encircling the formerly black center is significantly reduced, softened and flattened after ten days of once daily treatment.

Dry skin and calluses are softened by application of compositions to the whole foot. In one instance a lesion on a foot was characterized by deep fissures in the skin before treatment. Treatment with a sodium chlorite and clotrimazole composition followed by olive oil in a 1:2 ratio was applied once per day for two days. Three more treatments consisting of 2% sodium chlorite solution and water followed by twice as much of a mixture of olive oil and grapeseed oil were applied every other day. The fissure closed entirely and did not recur.

In two instances, a subject's irritation from insect bites was relieved by application of sodium chlorite and activated carbon. Approximately 12 hours after being bitten by a small flying insect (species undetermined) a bulls-eye inflammation pattern resulted that was 2.25 inches in diameter. Two drops of 2% Sodium chlorite solution were dropped onto activated carbon powder to form a paste. A quarter sized amount was applied to central bite area. Itching ceased immediately. A band-aid was next applied. Ninety minutes later the wound site was cleansed. Red inflamed “bull's eye pattern was entirely gone leaving a slightly raised bump about 6 mm in diameter. The next morning treatment was repeated. Poultice was removed after 45 minutes.

A spider bite was also treated with a combination of sodium chlorite and activated carbon in a beeswax mixture containing 3% thymol. Immediate cessation of itching is reported along with marked reduction in inflammation resulted.

Composition K appears to be useful to accelerate healing from injuries commonly characterized as cuts.

Composition 11 was applied to 2 keratoses lesions that had a rough dark appearance. Rough texture was eliminated. Dark Keratoses was lightened after 3 weeks of treatment.

Preliminary testing indicates that compositions described herein are effective for use in the mouth against tonsil stones, breath disorders, plaque and disease caused by plaque. Prior to treatment, subject had not had teeth professionally cleaned by a dentist in over 18 months and had severe plaque build-up. Subject brushed twice daily with Crest toothpaste. Periodontal disease of teeth #9, 10, 11 was diagnosed and treated two months previous to treatment with compositions described herein. A tooth cleaning was urgently recommended by subject's dentist.

A nickel-sized dollop of Composition K ointment was applied to the toothbrush. Next 1-2 drops of sodium chlorite was added to the mixture on the brush. Subject brushed teeth once daily for eleven days, augmenting in the evening with Crest. After eleven days, dentist evaluated results and concluded that “there is no sign of plaque wherever a tooth brush has touched the teeth.” The dentist did note a fine line of plaque on the back of the lower front incisors. Subject applied Composition K by mixing ointment and sodium chlorite in hand briefly, then applying liberally to gum line behind lower front incisors (#6-10). Treatment was repeated at least three times, no more than five times. Dentist again evaluated teeth for presence of tartar on the dorsal side of the lower front incisors. No evidence of tartar was detected upon inspection.

Composition b described in Zd was used to whiten front teeth. It was brushed on teeth numbered 5-10, upper and lower immediately after brushing with Composition K+1-2 drops sodium chlorite added to the brush. Teeth whitened dramatically after only three uses.

4) Subject L enthusiastically reported, and it was observed, that his toes changed color with treatment from grey to pink.

5) Acceleration of nail growth has been reported by 4 subjects regarding nail treatments that contain sodium chlorite in a several step system or one step system of application.

Subject K's left great toenail had been almost completely torn off. Her podiatrist advised that the nail be removed to prevent Onychomycosis. Subject did not have nail removed but continued treatment for 7 months with 2% sodium chlorite and olive oil, and compositions including thymol and menthol. (a 3 week treatment with sodium chlorite and 2% Miconazole was interspersed in the regimen) As expected, the severely damaged toenail fell off within 3 months revealing clear new nail growth. (A ¼ cm sliver of onychomycosis infected nail surmounted ½ cm of clear new growth indicating a section of nail that was present after the nail fell off). Subsequent treatment with Compositions M1 and K removed visual signs of fungus. It is beneficial to keep nail as long as possible after traumatic injury to maintain aesthetically appealing contours during grow-out. It is customary to remove nail after injury to prevent onychomycosis. The regimen described is an improvement over standard treatment of nail injury. Surprisingly, 85-90% of the toenail grew out in 6 months. This is considerably faster than the 12-16 months expected for a great toe to grow out. Subject J2, who characterized his nail growth before treatment as slow has grown out ½ of each toenail 2-5 in only two months. The new nail growth is clear of fungus. This indicates a treatment for diabetics who suffer from loss of circulation to feet and thus poor nail growth. It is also a promising adjunct therapy for those using ingested medications to treat onychomycosis.

What is claimed is:

1: A kit having at least two components, a first component being a solution containing a metal salt of chlorite and a second component containing an antimicrobial essential oil.

2: The kit of claim 1 wherein said second component contains at least one oil selected from the group consisting of thymol, menthol, menthyl acetate, olive oil, grape seed oil and olive oil.

3. The method of claim 1 further comprising a pharmaceutical antifungal agent.

4. A method of treating diseased or damaged integument comprising the steps of (1) applying a solution containing a metal salt of chlorite and (2) applying a composition containing an antimicrobial essential oil, wherein the steps may be in either order.

7. The method of claim 4 further comprising a pharmaceutical antifungal agent.

8. The method of claim 4 wherein the antimicrobial essential oil second component contains at least one oil selected from the group consisting of thymol, menthol, menthyl acetate, olive oil, grape seed oil and olive oil.
